(19) United States
(12) Patent Application Publication
(10) Pub. No.: US 2023/0279408 A1
(43) Pub. Date: Sep. 7, 2023

(54) COMPOSITIONS AND METHODS FOR INCREASING PROTEIN EXPRESSION

(71) Applicant: The Hong Kong University of Science and Technology, Kowloon (CN)

(72) Inventors: Yi Kuang, Kowloon (CN); Cheuk Yin Li, Kowloon (CN); Zhenghua Liang, Kowloon (CN); Kharis Daniel Setiasabda, Kowloon (CN)

(21) Appl. No.: 18/020,029

(22) PCT Filed: Aug. 6, 2021
(86) PCT No.: PCT/CN2021/111092

§ 371 Date: Feb. 6, 2023

Related U.S. Application Data
(60) Provisional application No. 63/103,471, filed on Aug. 7, 2020.

(51) Int. Cl. C12N 15/67

(52) U.S. Cl. CPC C12N 15/67; C12N 2830/50

(57) ABSTRACT

Provided are compositions and methods comprising artificial poly (A) sequences having at least one cytosine in the last one-third portion of the artificial poly (A) sequence closest to its 3′ end.

### RELATED APPLICATIONS

This application claims priority to U.S. Provisional Patent Application No. 63/103,471, filed Aug. 7, 2020, the contents of which is hereby incorporated by reference in the entirety for all purposes.

### BACKGROUND

Messenger RNA (mRNA) is the key molecule in the flow of genetic information. mRNAs are long nucleotide chains that encode protein information from the genome. They produce all the proteins in the cell, thus are one of the essential biomolecules of life. While mRNAs have been the subject of basic biological research for half a century, only in the past two decades has it been recognized and developed to be a potentially new powerful therapeutic tool [1]. Synthetic mRNA therapeutics, aka mRNA drugs, have several advantages over DNA- and protein-based counterparts [2]. mRNA possesses no risk of genomic integration as it is readily processed in the cytoplasm and does not enter the nucleus. It is also completely degraded by endogenous physiological metabolic pathways, allowing transient effect that is advantageous for pharmaceuticals [3]. Furthermore, mRNA naturally possesses sensory units, allowing it to tune protein production according to biomolecules present in the cell. In 1990, Wolff, et al. [4] demonstrated that injection of engineered mRNA on mice for in vivo expression of the encoded protein. This discovery led many research groups in the 1990s to explore the diverse applications of mRNA for biomedical purposes, such as gene therapy and vaccination [4-8]. While the results were promising, mRNA therapeutics face concerns in regard to its instability and high immunogenicity [9]. As mRNAs naturally degrades in the biological system, high dose or repeated administration is commonly required. There are artificial sequences and chemically modified nucleotides that can enhance mRNAs performance if placed in UTR and/or ORF sequences. There is a need for compositions and methods that can improve the stability of mRNAs.

### BRIEF SUMMARY

In one aspect, the disclosure features an artificial poly(A) sequence comprising about a string of about 30-150 consecutive (e.g., about 30, 35, 40, 45, 50, 55, 60, 65, 70, 75, 80, 85, 90, 95, 100, 105, 110, 115, 120, 125, 130, 135, 140, 145, or 150) adenines, wherein at least one adenine is substituted with a cytosine in the last one-third portion of the artificial poly(A) sequence closest to its 3′ end. In some embodiments, the artificial poly(A) sequence comprises between 18 and 149 (e.g., between 18 and 120, between 18 and 110, between 18 and 100, between 18 and 90, between 18 and 80, between 18 and 70, between 18 and 60, between 18 and 50, between 18 and 40, between 18 and 30, between 18 and 20, between 30 and 129, between 40 and 129, between 50 and 129, between 60 and 129, between 70 and 129, between 80 and 129, between 90 and 129, between 100 and 129, between 110 and 129, between 120 and 129, between 130 and 139, between 140 and 149) consecutive adenines, with at least one, possibly more, of which substituted with cytosine. In some embodiments, the last nucleotide of the artificial poly(A) sequence is not a cytosine.

In some embodiments, up to 40% (e.g., 2%, 4%, 6%, 8%, 10%, 12%, 14%, 16%, 18%, 20%, 22%, 24%, 26%, 28%, 30%, 32%, 34%, 36%, 38%, or 40%) of the nucleotides in the artificial poly(A) sequence are cytosines. In some embodiments, up to 25% (e.g., 2%, 4%, 6%, 8%, 10%, 12%, 14%, 16%, 18%, 20%, 22%, or 24%) of the nucleotides in the artificial poly(A) sequence are cytosines.

In some embodiments, most of the cytosines (i.e., 90% or more of the cytosines) in the artificial poly(A) sequence are located in the last one-third portion of the artificial poly(A) sequence closest to its 3′ end. Further, in some embodiments, all of the cytosines in the artificial poly(A) sequence are located consecutively.

In particular embodiments, the artificial poly(A) sequence comprises about 40 adenines and at least one adenine is substituted with a cytosine between the 27th nucleotide and the 39th nucleotide of the artificial poly(A) sequence. In certain embodiments, the artificial poly(A) sequence comprises between 24 and 39 (e.g., 24, 25, 26, 27, 28, 29, 30, 31, 32, 33, 34, 35, 36, 37, 38, or 39) adenines. In certain embodiments, the artificial poly(A) sequence comprises between 1 and 16 (e.g., 1, 2, 3, 4, 5, 6, 7, 8, 9, 10, 11, 12, 13, 14, 15, or 16) cytosines. In some embodiments, all of the cytosines in the artificial poly(A) sequence are located between the 25th nucleotide and the 39th nucleotide of the artificial poly(A) sequence. Further, in certain embodiments, all of the cytosines in the artificial poly(A) sequence are located consecutively. In some embodiments, the last nucleotide of the artificial poly(A) sequence is not a cytosine.

In particular embodiments, the artificial poly(A) sequence comprises about 60 adenines and at least one adenine is substituted with a cytosine between the 41th nucleotide and the 59th nucleotide of the artificial poly(A) sequence. In certain embodiments, the artificial poly(A) sequence comprises between 36 and 59 (e.g., 36, 37, 38, 39, 40, 41, 42, 43, 44, 45, 46, 47, 48, 49, 50, 51, 52, 53, 54, 55, 56, 57, 58, or 59) adenines. In certain embodiments, the artificial poly(A) sequence comprises between 1 and 24 (e.g., 1, 2, 3, 4, 5, 6, 7, 8, 9, 10, 11, 12, 13, 14, 15, 16, 17, 18, 19, 20, 21, 22, 23, or 24) cytosines. In some embodiments, all of the cytosines in the artificial poly(A) sequence are located between the 37th nucleotide and the 59th nucleotide of the artificial poly(A) sequence. Further, in certain embodiments, all of the cytosines in the artificial poly(A) sequence are located consecutively. In some embodiments, the last nucleotide of the artificial poly(A) sequence is not a cytosine.

In particular embodiments, the artificial poly(A) sequence comprises about 100 adenines and at least one adenine is substituted with a cytosine between the 67^{th} nucleotide and the 99^{th} nucleotide of the artificial poly(A) sequence. In certain embodiments, the artificial poly(A) sequence comprises between 60 and 99 (e.g., 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99) adenines. In certain embodiments, the artificial poly(A) sequence comprises between 1 and 40 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40) cytosines. In some embodiments, all of the cytosines in the artificial poly(A) sequence are located between the 61^{st} nucleotide and the 99^{th} nucleotide of the artificial poly(A) sequence. Further, in some embodiments, all of the cytosines in the artificial poly(A) sequence are located consecutively. In some embodiments, the last nucleotide of the artificial poly(A) sequence is not a cytosine. The claimed poly(A) sequence of this invention is able to, when present at the 3′ end of a polypeptide-encoding sequence in an mRNA molecule, improve the stability of the mRNA. Further improvement of stability is achieved synergistically by way of additional modification of the mRNA including 5′cap modification, artificial 5′ and 3′ UTR sequences, and a coding region with an optimized codon, as well as chemical modifications of the mRNA such as the substitution of naturally-occurring nucleotides with non-naturally-occurring nucleotides, e.g., pseudouridine and 5-methyl-cytosine.

The disclosure also features an expression cassette comprising a promoter and a nucleotide sequence encoding the artificial poly(A) sequence described herein. In some embodiments, the expression cassette further comprises a multiple cloning site between the promoter and the coding sequence for the artificial poly(A) sequence so as to permit insertion of a polynucleotide sequence encoding a protein of interest to be operably linked to the promoter and the sequence encoding the poly(A) sequence. In some embodiments, the expression cassette further comprises a transcription initiation codon and a transcription termination codon, both operably linked to the promoter and the sequence encoding the artificial poly(A) sequence. In particular embodiments, the expression cassette further comprises a polynucleotide sequence encoding a polypeptide between the promoter and the sequence encoding the artificial poly(A) sequence, wherein the polynucleotide sequence is operably linked to the promoter and the sequence encoding the artificial poly(A) sequence.

The disclosure also provides an expression vector (e.g., a circularized vector such as a plasmid or a viral vector) comprising the expression cassette described herein.

In another aspect, the disclosure also provides a host cell comprising the expression cassette or the expression vector described herein.

In another aspect, the disclosure provides an RNA polynucleotide expressed from the expression cassette described herein as well as an RNA molecule that contains, from 5′ end to 3′ end, a polynucleotide sequence encoding a polypeptide and a poly(A) sequence of this invention as described above and herein.

In a further aspect, the disclosure provides a method of increasing protein expression of a polypeptide inside a cell, comprising transfecting the cell with the expression vector described herein.

### BRIEF DESCRIPTION OF THE DRAWINGS

FIGS. 1A and 1B show the influence of single nucleotide substitution at and near the end of poly(A) tail towards the protein expression of synthetic EGFP mRNAs on HEK293 cells. 4, 24, 48 and 72 hours after transfection, the mean EGFP signals from positively transfected cells were recorded for analysis. (A) Time-dependent EGFP expression curve on HEK293 cells. (B) Comparison of the EGFP expression at 24 hours after transfection. (n=3; data presented at mean±standard deviation). *=P<0.05; one-way ANOVA.

FIG. 2 shows the influence of dual cytidine substitutions near the end of poly(A) tail towards the protein expression of synthetic EGFP mRNA on HEK293 cells. 4 and 24 hours after transfection, the mean EGFP expression from positively transfected cells were recorded for analysis (n=3; data presented at mean±standard deviation). *=P<0.05; one-way ANOVA.

FIG. 3 shows the effect of cytidine substitution on poly(A) tail towards the protein expression of mRNA on various types of cells. 24 hours after transfection, the mean EGFP expression from positively transfected cells were recorded for analysis. The EGFP expression from cells transfected with EGFP mRNAs with 40A poly(A) tail serves as reference (n=3; data presented at mean±standard deviation). *=P<0.05; **=P<0.01; ***=P<0.001; one-way ANOVA.

FIGS. 4A and 4B show the correlation between cytidine substitution frequency on poly(A) tail and protein expression efficiency of EGFP mRNAs. (A) Comparison of relative EGFP expression at 24 hours after transfection. (B) Relative EGFP expression 24, 48, and 72 hours after transfection. The mean EGFP expression from positively transfected HEK293 cells are recorded for analysis. The EGFP expression from cells transfected with EGFP mRNA with 40A poly(A) tail serves as reference (n=3; data presented at mean±standard deviation). *=P<0.05; **=P<0.01; ***=P<0.001; one-way ANOVA.

FIG. 5 shows the correlation between cytidine substitution location on poly(A) tail and protein expression efficiency of EGFP mRNAs. 24 hours after transfection, the mean EGFP expression from positively transfected HEK293 cells were recorded for analysis. The EGFP expression from cells transfected with EGFP mRNA with 40A poly(A) tail serves as reference (n=3; data presented at mean±standard deviation). *=P<0.05; one-way ANOVA.

FIG. 6 shows the effect of cytidine substitution on poly(A) tail to the enzymatic activities of SEAP-coding model mRNAs. The SEAP expression from HEK293 cells were recorded by measuring the activity of SEAP in the culture media collected by complete medium replacement at 24, 48 and 72 hours after transfection (n=3; data presented at mean±standard deviation). *=P<0.05; **=P<0.01; ***=P<0.001; one-way ANOVA.

FIGS. 7A and 7B show the effect of cytidine substitution on poly(A) tail to the performance of microRNA-sensing smart mRNAs with different poly(A) tails. HEK293 and HeLa cells, having low and high expression of microRNA-21-5p, were seeded as a near equal mixture. 24 hours after transfection, a high EGFP expression population (corresponding to HEK293) and a low EGFP expression population (corresponding to HeLa) were selected on the flow cytometer using the same gate setting. The difference in adjusted mean EGFP expression from these two populations represents the precision of targeted delivery by the smart mRNAs. (A) scheme of the smart mRNA. (B) the precision level between smart mRNAs with 40A and 37ACCA tails (n=3; data presented at mean±standard deviation). *=P<0.05; paired-T test.

FIGS. 8A-8C show that poly(A) tails with cytidine substitution prolong the half-lives of synthetic mRNAs inside the cells. (A) Cell viability and (B) transfection efficiency was recorded after 24 hours of transfection of EGFP mRNAs with different poly(A) tails (n=3; data presented at mean±standard deviation). (C) RT-qPCR was used to quantify the amount of intracellular EGFP mRNAs at different time points after transfection. The 18S rRNA mRNAs were used for normalization. The value of EGFP mRNA with 40A poly(A) tail at 4 hours is set as 1 (n=3; data presented at mean±standard deviation). *=P<0.05; one-way ANOVA.

FIG. 9 shows that the effect of cytosine substitution on increasing protein expression works with poly(A) tails of different lengths (n=3; data presented at mean±standard deviation). *=P<0.05; **=P<0.01; ***=P<0.001; one-way ANOVA.

FIG. 10 shows that lower concentration of mRNA with C tail (EGFP-31A8CA mRNA) can achieve similar or higher protein expression level as mRNA with A-only tail (EGFP-40A mRNA) (n=3; data presented at mean±standard deviation).

FIGS. 11A and 111B show the effect of C tails induced protein expression enhancement being independent of the transfection reagent. Enhancement of EGFP expression from EGFP mRNAs with C containing tails were observed using (A) a polymer-based reagent PEI-Transferrinfection kit (1.5 μL of 1 mg/mL PEI per well in 48 well plate) or (B) a lipofectamine 3000 reagent (0.5 μL of lipofectamine 3000 per well in 48 well plate) to deliver the mRNAs to HEK293 cells (n=3; data are presented as mean±SD).

FIG. 12 shows the EGFP expression of EGFP mRNAs carrying different tails in HeLa cytoplasmic extract over time (IpraCell). The protein production enhancement effect of C containing tails also worked in in vitro setting (n=3; data are presented as mean±SD).

FIGS. 13A and 13B show the C containing tails can be used with existing mRNA enhancement technologies. (A) EGFP mRNAs with different tails were synthesized using 100% substitution of cytidine by 5 methylcytidine. Same enhancement effect of EGFP production by the C tails was observed after 24 hours of mRNA transfection on HEK293 cells. (B) EGFP mRNAs were synthesized to carry: a weak m₃^{2.2.7}GP₃G cap (−) or a strong ARCA cap (+); canonical nucleotides (−) or 100% substitution of uridine by N¹-methylpseudouridine (+); 40A tail (−) or 31A8CA tail (+). ARCA cap and N¹-methylpseudouridine both can enhance protein production of mRNA, as shown in prior arts. The EGFP mRNAs were transfected to HEK293 cells and synergic protein production enhancement effect by the C containing tails with modified cap or with modified nucleotide and modified cap were observed after 24 hours of mRNA transfection (n=3; data are presented as mean±SD).

FIG. 14 shows in the case of multiple C substitution, having the terminal nucleotide as C in the tail shows weak protein production effect. EGFP-38ACC mRNA exhibited slight enhancement in EGFP expression than EGPF-40A mRNA from HEK293 cells after 24 hours of mRNA transfection.

### DETAILED DESCRIPTION

### I. Introduction

The inventors have discovered that artificial poly(A) sequences containing adenines and at least one cytosine, when joined to the 3′ end of an RNA sequence, can effectively enhance protein expression from the RNA sequence. These artificial poly(A) sequences can be used for both simple and smart model mRNA drugs, with the effect being cell type independent and delivery reagent independent. As the artificial poly(A) sequences can be simply incorporated into the DNA templates by regular PCR reactions, no additional cost is needed for synthesizing mRNA drugs carrying the artificial poly(A) sequences. The artificial poly(A) sequence can be used with other mRNA technologies including modified nucleotide, modified cap analog. Therefore, these artificial poly(A) sequences can be broadly used on the existing and future mRNA drugs for enhancement of efficacy and for reduction of cost.

### II. Definitions

As used herein, the term “artificial poly(A) sequence” refers to an RNA polynucleotide containing a string of consecutive adenines, among which at least one is substituted with cytosine. Typically, the last nucleotide in the artificial poly(A) sequence is not cytosine.

As used herein, the phrase “last one-third portion of the artificial poly(A) sequence closest to its 3′ end” refers to the nucleotides located close to the 3′ end of the artificial poly(A) sequence, in which these nucleotides make up one-third of all the nucleotides in the sequence. For example if the artificial poly(A) sequence has 40 nucleotides, the last one-third portion of the artificial poly(A) sequence closest to its 3′ end refers to the 27^{th} nucleotide to the 40^{th} nucleotide. In another example if the artificial poly(A) sequence has 20 nucleotides, the last one-third portion of the artificial poly(A) sequence closest to its 3′ end refers to the 14^{th} nucleotide to the 20^{th} nucleotide.

As used herein, the term “about” denotes a range of values that is +/−10% of a specified value. For instance, “about 40” denotes the value range of 40+/−40×10%, i.e., 36 to 44.

As used herein, the term “between” denotes a range of values set within a lower bound and an upper bound, in which the lower bound value and the upper bound value are included. For example, a nucleotide between the 27^{th} nucleotide and the 39^{th} nucleotide, of a polynucleotide containing total 40 nucleotides, can be the 27^{th}, 28^{th}, 29^{th}, 30^{th}, 31^{st}, 32^{rd}, 33^{rd} 34^{rd}, 35^{th}, 36^{th}, 37^{th}, 38^{th}, or 39^{th} nucleotide.

The term “expression cassette” refers to a nucleic acid construct, generated recombinantly or synthetically, with a series of specified nucleic acid elements that permit transcription of a particular polynucleotide sequence in a host cell. An expression cassette may be a part of a circular construct such as a plasmid, a viral genome or vector, or a longer nucleic acid fragment. Typically, an expression cassette includes a polynucleotide to be transcribed, operably linked to a promoter (e.g., a heterologous promoter). “Operably linked” in this context means that two or more genetic elements, such as a polynucleotide coding sequence and a promoter, are placed in relative positions that permit the proper biological functioning of the elements, such as the promoter directing transcription of the coding sequence. Other elements (e.g., heterologous elements) that may be present in an expression cassette include those that enhance transcription (e.g., enhancers) and terminate transcription (e.g., terminators), as well as those that confer certain binding affinity or antigenicity to the recombinant protein produced from the expression cassette.

The term “multiple cloning site” refers to a short stretch of nucleotide sequence comprising multiple restriction endonuclease recognition sites permitting insertion of another sequence encoding an RNA or protein.

The term “nucleic acid” refers to deoxyribonucleotides or ribonucleotides and polymers thereof in either single- or double-stranded form, anxd complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

As used herein, the term “polynucleotide” refers to an oligonucleotide, or nucleotide, and fragments or portions thereof, and to DNA or RNA of genomic or synthetic origin, which may be single- or double-stranded, and represent the sense or anti-sense strand. A single polynucleotide is translated into a single polypeptide.

As used herein, the terms “peptide” and “polypeptide” are used interchangeably and describe a single polymer in which the monomers are amino acid residues which are joined together through amide bonds. A polypeptide is intended to encompass any amino acid sequence, either naturally occurring, recombinant, or synthetically produced.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST 2.0 sequence comparison algorithms with default parameters described below, or by manual alignment and visual inspection (see, e.g., NCBI web site ncbi.nlm.nih.gov/BLAST/ or the like). Such sequences are then said to be “substantially identical.” As described below, the preferred algorithms can account for gaps and the like. Preferably, identity exists over a region that is at least about 25 amino acids or nucleotides in length, or more preferably over a region that is 50-100 or more amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art.

An algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the disclosure. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

### III. Artificial Poly(A) Sequence

The disclosure provides an artificial poly(A) sequence that has at least one cytosine. The artificial poly(A) sequence can contain about 30-130 adenines, in which at least one adenine is substituted with a cytosine in the last one-third portion of the artificial poly(A) sequence closest to its 3′ end. In certain embodiments, the artificial poly(A) sequence can contain 30, 31, 32, 33, 34, 35, 36, 37, 38, 39, 40, 41, 42, 43, 44, 45, 46, 47, 48, 49, or 50 adenines, in which at least one adenine in the last one-third portion of the artificial poly(A) sequence closest to its 3′ end is substituted with a cytosine. In other embodiments, two or more adenines in the artificial poly(A) sequence are substituted with cytosines and at least one cytosine is located in the last one-third portion of the artificial poly(A) sequence closest to its 3′ end. In some embodiments of the artificial poly(A) sequence, up to 40% (e.g., 2%, 4%, 6%, 8%, 10%, 12%, 14%, 16%, 18%, 20%, 22%, 24%, 26%, 28%, 30%, 32%, 34%, 36%, 38%, or 40%) of the nucleotides in the artificial poly(A) sequence are cytosines. In some embodiments of the artificial poly(A) sequence, from 60% to 98% (e.g., 60%, 62%, 64%, 66%, 68%, 70%, 72%, 74%, 76%, 78%, 80%, 82%, 84%, 86%, 88%, 90%, 92%, 94%, 96%, or 98%) of the nucleotides in the artificial poly(A) sequence are adenines. In some embodiments, the artificial poly(A) sequence can contain between 18 and 129 (e.g., between 18 and 120, between 18 and 110, between 18 and 100, between 18 and 90, between 18 and 80, between 18 and 70, between 18 and 60, between 18 and 50, between 18 and 40, between 18 and 30, between 18 and 20, between 30 and 129, between 40 and 129, between 50 and 129, between 60 and 129, between 70 and 129, between 80 and 129, between 90 and 129, between 100 and 129, between 110 and 129, between 120 and 129) adenines. In some embodiments, the artificial poly(A) sequence can contain between 1 and 20 (e.g., 1, 2, 3, 4, 5, 6, 7, 8, 9, 10, 11, 12, 13, 14, 15, 16, 17, 18, 19, or 20) cytosines. In some embodiments of the artificial poly(A) sequences described herein, the last nucleotide of the artificial poly(A) sequence is not a cytosine.

In certain embodiments of the artificial poly(A) sequences described herein, most of the cytosines (i.e., 90% or more of the cytosines) in the artificial poly(A) sequence are located in the last one-third portion of the artificial poly(A) sequence closest to its 3′ end. The cytosines in the artificial poly(A) sequence can be located consecutively, i.e., in a contiguous chain of cytosines without any adenines in between. In some embodiments, the cytosines in the artificial poly(A) sequence can be located consecutively in the last one-third portion of the artificial poly(A) sequence closest to its 3′ end, in which the last nucleotide in the artificial poly(A) sequence is not cytosine. In other embodiments, the cytosines in the artificial poly(A) sequence can be spread out (i.e., adenines may be located between cytosines) throughout the length of the artificial poly(A) sequence. In some embodiments, the cytosines in the artificial poly(A) sequence can be spread out within in the last one-third portion of the artificial poly(A) sequence closest to its 3′ end, in which the last nucleotide in the artificial poly(A) sequence is not cytosine.

In particular, an artificial poly(A) sequence can contain about 40 adenines and at least one adenine is substituted with a cytosine between the 27^{th} nucleotide and the 39^{th} nucleotide of the artificial poly(A) sequence, in which the last nucleotide of the artificial poly(A) sequence is not a cytosine. In some embodiments of this artificial poly(A) sequence, the sequence can contain between 1 and 16 (e.g., 1, 2, 3, 4, 5, 6, 7, 8, 9, 10, 11, 12, 13, 14, 15, or 16) cytosines. In certain embodiments of this artificial poly(A) sequence, the sequence can contain between 24 and 39 (e.g., 24, 25, 26, 27, 28, 29, 30, 31, 32, 33, 34, 35, 36, 37, 38, or 39) adenines. In certain embodiments of this artificial poly(A) sequence, all of the cytosines in the artificial poly(A) sequence are located between the 25^{th} nucleotide and the 39^{th} nucleotide (e.g., between the 26^{th} and the 39^{th} nucleotide, between the 27^{th} and the 39^{th} nucleotide, between the 28^{th} and the 39^{th} nucleotide, between the 29^{th} and the 39^{th} nucleotide, between the 30^{th} and the 39^{th} nucleotide, between the 31^{st} and the 39^{th} nucleotide, between the 32^{nd} and the 39^{th} nucleotide, between the 33^{rd} and the 39^{th} nucleotide, between the 34^{th} and the 39^{th} nucleotide, between the 35^{th} and the 39^{th} nucleotide, between the 36^{th} and the 39^{th} nucleotide, or between the 37^{th} and the 39^{th} nucleotide) of the artificial poly(A) sequence. In certain embodiments, all of the cytosines in the artificial poly(A) sequence are located consecutively, i.e., in a contiguous chain of cytosines without any adenines in between. In certain embodiments, all of the cytosines in the artificial poly(A) sequence are located consecutively between the 25^{th} nucleotide and the 39^{th} nucleotide (e.g., between the 26^{th} and the 39^{th} nucleotide, between the 27^{th} and the 39^{th} nucleotide, between the 28^{th} and the 39^{th} nucleotide, between the 29^{th} and the 39^{th} nucleotide, between the 30^{th} and the 39^{th} nucleotide, between the 31^{st} and the 39^{th} nucleotide, between the 32^{nd} and the 39^{th} nucleotide, between the 33^{rd} and the 39^{th} nucleotide, between the 34^{th} and the 39^{th} nucleotide, between the 35^{th} and the 39^{th} nucleotide, between the 36^{th} and the 39^{th}nucleotide, or between the 37^{th} and the 39^{th} nucleotide) of the artificial poly(A) sequence, in which the last nucleotide in the artificial poly(A) sequence is not cytosine.

In particular, an artificial poly(A) sequence can contain about 60 adenines and at least one adenine is substituted with a cytosine between the 41st nucleotide and the 59^{th} nucleotide of the artificial poly(A) sequence, in which the last nucleotide of the artificial poly(A) sequence is not a cytosine. In some embodiments of this artificial poly(A) sequence, the sequence can contain between 1 and 24 (e.g., 1, 2, 3, 4, 5, 6, 7, 8, 9, 10, 11, 12, 13, 14, 15, 16, 17, 18, 19, 20, 21, 22, 23, or 24) cytosines. In certain embodiments of this artificial poly(A) sequence, the sequence can contain between 36 and 59 (e.g., 36, 37, 38, 39, 40, 41, 42, 43, 44, 45, 46, 47, 48, 49, 50, 51, 52, 53, 54, 55, 56, 57, 58, or 59) adenines. In certain embodiments of this artificial poly(A) sequence, all of the cytosines in the artificial poly(A) sequence are located between the 37th nucleotide and the 59th nucleotide (e.g., between the 38th and the 59th nucleotide, between the 39th and the 59th nucleotide, between the 40th and the 59th nucleotide, between the 41st and the 59th nucleotide, between the 42nd and the 59th nucleotide, between the 43rd and the 59th nucleotide, between the 44th and the 59th nucleotide, between the 45th and the 59th nucleotide, between the 46th and the 59th nucleotide, between the 47th and the 59th nucleotide, between the 48th and the 59th nucleotide, between the 49th and the 59th nucleotide, between the 50th and the 59th nucleotide, between the 51st and the 59th nucleotide, between the 52nd and the 59th nucleotide, between the 53rd and the 59th nucleotide, between the 54th and the 59th nucleotide, between the 55th and the 59th nucleotide, between the 56th and the 59th nucleotide, or between the 57th and the 59th nucleotide) of the artificial poly(A) sequence. In certain embodiments, all of the cytosines in the artificial poly(A) sequence are located consecutively, i.e., in a contiguous chain of cytosines without any adenines in between. In certain embodiments, all of the cytosines in the artificial poly(A) sequence are located consecutively between the 37th nucleotide and the 59th nucleotide (e.g., between the 38th and the 59th nucleotide, between the 39th and the 59th nucleotide, between the 40th and the 59th nucleotide, between the 41st and the 59th nucleotide, between the 42nd and the 59th nucleotide, between the 43rd and the 59th nucleotide, between the 44th and the 59th nucleotide, between the 45th and the 59th nucleotide, between the 46th and the 59th nucleotide, between the 47th and the 59th nucleotide, between the 48th and the 59th nucleotide, between the 49th and the 59th nucleotide, between the 50th and the 59th nucleotide, between the 51st and the 59th nucleotide, between the 52nd and the 59th nucleotide, between the 53rd and the 59th nucleotide, between the 54th and the 59th nucleotide, between the 55th and the 59th nucleotide, between the 56th and the 59th nucleotide, or between the 57th and the 59th nucleotide) of the artificial poly(A) sequence, in which the last nucleotide in the artificial poly(A) sequence is not cytosine.

In particular, an artificial poly(A) sequence can contain about 100 adenines and at least one adenine is substituted with a cytosine between the 67th nucleotide and the 99th nucleotide of the artificial poly(A) sequence, in which the last nucleotide of the artificial poly(A) sequence is not a cytosine. In some embodiments of this artificial poly(A) sequence, the sequence can contain between 1 and 40 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40) cytosines. In certain embodiments of this artificial poly(A) sequence, the sequence can contain between 60 and 99 (e.g., 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99) adenines. In certain embodiments of this artificial poly(A) sequence, all of the cytosines in the artificial poly(A) sequence are located between the 61st nucleotide and the 99th nucleotide (e.g., between the 62nd and the 99th nucleotide, between the 63rd and the 99th nucleotide, between the 64th and the 99th nucleotide, between the 65st and the 99th nucleotide, between the 66th and the 99th nucleotide, between the 67th and the 99th nucleotide, between the 68th and the 99th nucleotide, between the 69th and the 99th nucleotide, between the 70th and the 99th nucleotide, between the 71st and the 99th nucleotide, between the 72nd and the 99th nucleotide, between the 73rd and the 99th nucleotide, between the 74th and the 99th nucleotide, between the 75th and the 99th nucleotide, between the 76th and the 99th nucleotide, between the 77th and the 99th nucleotide, between the 78th and the 99th nucleotide, between the 79th and the 99th nucleotide, between the 80th and the 99th nucleotide, between the 81st and the 99th nucleotide, between the 82nd and the 99th nucleotide, between the 83rd and the 99th nucleotide, between the 84th and the 99th nucleotide, between the 85th and the 99th nucleotide, between the 86th and the 99th nucleotide, between the 87th and the 99th nucleotide, between the 88th and the 99th nucleotide, between the 89th and the 99th nucleotide, between the 90th and the 99th nucleotide, between the 91st and the 99th nucleotide, between the 92nd and the 99th nucleotide, between the 93rd and the 99th nucleotide, between the 94th and the 99th nucleotide, between the 95th and the 99th nucleotide, between the 96th and the 99th nucleotide, or between the 97th and the 99th nucleotide) of the artificial poly(A) sequence. In certain embodiments, all of the cytosines in the artificial poly(A) sequence are located consecutively, i.e., in a contiguous chain of cytosines without any adenines in between. In certain embodiments, all of the cytosines in the artificial poly(A) sequence are located consecutively between the 61st nucleotide and the 99th nucleotide (e.g., between the 62nd and the 99th nucleotide, between the 63rd and the 99th nucleotide, between the 64th and the 99th nucleotide, between the 65st and the 99th nucleotide, between the 66th and the 99th nucleotide, between the 67th and the 99th nucleotide, between the 68th and the 99th nucleotide, between the 69th and the 99th nucleotide, between the 70th and the 99th nucleotide, between the 71st and the 99th nucleotide, between the 72nd and the 99th nucleotide, between the 73rd and the 99th nucleotide, between the 74th and the 99th nucleotide, between the 75th and the 99th nucleotide, between the 76th and the 99th nucleotide, between the 77th and the 99th nucleotide, between the 78th and the 99th nucleotide, between the 79th and the 99th nucleotide, between the 80th and the 99th nucleotide, between the 81st and the 99th nucleotide, between the 82nd and the 99th nucleotide, between the 83rd and the 99th nucleotide, between the 84th and the 99th nucleotide, between the 85th and the 99th nucleotide, between the 86th and the 99th nucleotide, between the 87th and the 99th nucleotide, between the 88th and the 99th nucleotide, between the 89th and the 99th nucleotide, between the 90th and the 99th nucleotide, between the 91st and the 99th nucleotide, between the 92nd and the 99th nucleotide, between the 93rd and the 99th nucleotide, between the 94th and the 99th nucleotide, between the 95th and the 99th nucleotide, between the 96th and the 99th nucleotide, or between the 97th and the 99th nucleotide) of the artificial poly(A) sequence, in which the last nucleotide in the artificial poly(A) sequence is not cytosine.

In other embodiments of this artificial poly(A) sequence, the cytosines in the artificial poly(A) sequence can be spread out (i.e., adenines may be located between cytosines) throughout the length of the artificial poly(A) sequence. In some embodiments, the cytosines in the artificial poly(A) sequence can be spread out between the 25^{th} nucleotide and the 39^{th} nucleotide (e.g., between the 26^{th} and the 39^{th} nucleotide, between the 27^{th} and the 39^{th} nucleotide, between the 28^{th} and the 39^{th} nucleotide, between the 29^{th} and the 39^{th} nucleotide, between the 30^{th} and the 39^{th} nucleotide, between the 31^{st} and the 39^{th} nucleotide, between the 32^{nd} and the 39^{th} nucleotide, between the 33^{rd} and the 39^{th} nucleotide, between the 34^{th} and the 39^{th} nucleotide, between the 35^{th} and the 39^{th} nucleotide, between the 36^{th} and the 39^{th} nucleotide, or between the 37^{th} and the 39^{th} nucleotide) of the artificial poly(A) sequence, in which the last nucleotide in the artificial poly(A) sequence is not cytosine.

In particular embodiments, the artificial poly(A) sequence described herein comprises a sequence having at least 90% (e.g., 90%, 91%, 92%, 93%, 94%, 95%, 96%, 97%, 98%, 99%, or 100%) identity to a sequence of any one of SEQ ID NOS:5 and 7-11.

### IV. Expression Cassette and Vector

The disclosure also provides expression cassettes comprising a promoter and an artificial poly(A) sequence described herein. Such an expression cassette, especially in the form of a replicable vector (e.g., a DNA plasmid or a viral vector), is useful tool for the cloning/subcloning and expression of any coding sequence for a protein. Thus, in some cases, the expression cassette can further comprise a polynucleotide sequence encoding a polypeptide between the promoter and the artificial poly(A) sequence, wherein the polynucleotide sequence is operably linked to the promoter and the artificial poly(A) sequence. In some embodiments, the expression cassette can further comprise a multiple cloning site between the promoter and the artificial poly(A) sequence. Moreover, the expression cassette can further comprise a transcription initiation codon and a transcription termination codon, both of which can be operably linked to the promoter and the artificial poly(A) sequence. Additional elements such as transcriptional activation or enhancer sequences may be included in the expression cassettes and vectors.

In some embodiments, the promoter may be homologous or heterologous to the polynucleotide between the promoter and the artificial poly(A) sequence. In some embodiments, the promoter may be inducible. In some embodiments, the promoter may be cell or tissue-specific. In some embodiments, the promoter may be a constitutive promoter. In some embodiments, the expression cassette can be expressed specifically in certain cell and/or tissue types within one or more organs. Alternatively, the expression cassette can be expressed constitutively (e.g., using a constitutive promoter). Further, an expression cassette can contain a marker gene that confers a selectable phenotype on transfected cells. For example, the marker may encode antibiotic resistance, such as resistance to kanamycin, G418, bleomycin, or hygromycin.

The disclosure also provides expression vectors comprising the expression cassette. The expression vectors serve as vehicles that can deliver the expression cassettes into the targeted destination, e.g., inside cells. The expression vectors can be transfected into cells. Techniques for transfecting a wide variety of cells are well known and described in the technical and scientific literature. See, e.g., Kim and Eberwine, Anal Bioanal Chem. 397(8):3173-8, 2020. The disclosure also provides a host cell that comprises the expression cassette or the expression vector described herein. Once transfected into the target cells, the polynucleotide encoding the polypeptide and the artificial poly(A) sequence can be transcribed into an RNA polynucleotide.

### V. Other Modifications

An artificial poly(A) sequence described herein or an RNA polynucleotide containing an artificial poly(A) sequence described herein can contain other modifications to improve its stability.

To address its issues, modifications of mRNA structural elements have been investigated to improve the stability and translational efficiency. These modifications include 5′cap modification, artificial 5′ and 3′ UTR sequences, and a coding region with an optimized codon [1, 10, 11]. Furthermore, chemical modifications of mRNA molecules, including pseudouridine and 5-methyl-cytosine, have been observed to increase protein translation while reducing immune response [12-14].

Modified Nucleobases

An artificial poly(A) sequence described herein or an RNA polynucleotide containing an artificial poly(A) sequence described herein can contain one or more modified nucleobases. A modified nucleobase (or base) refers to a nucleobase having at least one change that is structurally distinguishable from a naturally-occurring nucleobase (i.e., adenine, guanine, cytosine, thymine, or uracil). In some embodiments, a modified nucleobase is functionally interchangeable with its naturally-occurring counterpart. Both naturally-occurring and modified nucleobases are capable of hydrogen bonding. Modified nucleobases may help to improve the stability of a polynucleotide, such as increasing its half-life and preventing intracellular degradation and proteolytic cleavage. In some embodiments, an artificial poly(A) sequence described herein or an RNA polynucleotide containing an artificial poly(A) sequence described herein may include at least one modified nucleobase. Examples of modified nucleobases include, but are not limited to, 5-methylcytosine, 5-hydroxymethyl cytosine, xanthine, hypoxanthine, 2-aminoadenine, 6-methyladenine, 6-methylguanine, 2-propyladenine, 2-propylguanine, 2-thiouracil, 2-thiothymine, 2-thiocytosine, 5-halouracil, 5-halocytosine, 5-propynyluracil, 5-propynylcytosine, 6-azouracil, 6-azocytosine, 6-azothymine, 5-uracil (pseudouracil), 4-thiouracil, 8-haloadenine, 8-aminoadenine, 8-thioladenine, 8-thioalkyladenine, 8-hydroxyladenine, 8-haloguanine, 8-aminoguanine, 8-thiolguanine, 8-thioalkylguanine, 8-hydroxylguanine, 5-halouracil, 5-bromouracil, 5-trifluoromethyluracil, 5-halocytosine, 5-bromocytosine, 5-trifluoromethylcytosine, 7-methylguanine, 7-methyladenine, 2-fluoroadenine, 2-aminoadenine, 8-azaguanine, 8-azaadenine, 7-deazaguanine, 7-deazaadenine, 3-deazaguanine, and 3-deazaadenine.

Modified Sugars

An artificial poly(A) sequence described herein or an RNA polynucleotide containing an artificial poly(A) sequence described herein can contain one or more modified sugars. A modified sugar refers to a sugar having at least one change that is structurally distinguishable from a naturally-occurring sugar (i.e., ribose in RNA). Modifications on modified sugars may help to improve the stability of an artificial poly(A) sequence described herein or an RNA polynucleotide containing an artificial poly(A) sequence described herein. In some embodiments, the sugar is a pentofuranosyl sugar. The pentofuranosyl sugar ring of a nucleoside may be modified in various ways including, but not limited to, addition of a substituent group, particularly, at the 2′ position of the ring; bridging two non-geminal ring atoms to form a bicyclic sugar (i.e., a locked sugar); and substitution of an atom or group such as —S—, —N(R)— or —C(R₁)(R₂) for the ring oxygen. Examples of modified sugars include, but are not limited to, substituted sugars, especially 2′-substituted sugars having a 2′-F, 2′-OCH₂ (2′-OMe), or a 2′-O(CH₂)₂—OCH₃ (2′-O-methoxyethyl or 2′-MOE) substituent group; and bicyclic sugars. A bicyclic sugar refers to a modified pentofuranosyl sugar containing two fused rings. For example, a bicyclic sugar may have the 2′ ring carbon of the pentofuranose linked to the 4′ ring carbon by way of one or more carbons (i.e., a methylene) and/or heteroatoms (i.e., sulfur, oxygen, or nitrogen). The second ring in the sugar limits the flexibility of the sugar ring and thus, constrains the oligonucleotide in a conformation that is favorable for base pairing interactions with its target nucleic acids. An example of a bicyclic sugar is a locked sugar, which is a pentofuranosyl sugar having the 2′-oxygen linked to the 4′ ring carbon by way of a carbon (i.e., a methylene) or a heteroatom (i.e., sulfur, oxygen, or nitrogen). In some embodiments, a locked sugar has the 2′-oxygen linked to the 4′ ring carbon by way of a carbon (i.e., a methylene). In other words, a locked sugar has a 4′-(CH₂)—O-2′ bridge, such as α-L-methyleneoxy (4′-CH₂—O-2′) and β-D-methyleneoxy (4′-CH₂—O-2′). A nucleoside having a lock sugar is referred to as a locked nucleoside.

Other examples of bicyclic sugars include, but are not limited to, (6'S)-6′ methyl bicyclic sugar, aminooxy (4′-CH₂—O—N(R)-2′) bicyclic sugar, oxyamino (4′-CH₂—N(R)—O-2′) bicyclic sugar, wherein R is, independently, H, a protecting group or C₁-C₁₂ alkyl. The substituent at the 2′ position can also be selected from allyl, amino, azido, thio, O-allyl, O—C1-C10 alkyl, OCF₃, O(CH₂)₂SCH₃, O(CH₂)₂—O—N(Rₘ)(Rₙ), and O—CH₂—C(═O)—N(Rₘ)(Rₙ), wherein each Rₘ and Rₙ is, independently, H or substituted or unsubstituted C1-C10 alkyl.

In some embodiments, a modified sugar is an unlocked sugar. An unlocked sugar refers to an acyclic sugar that has a 2′, 3′-seco acyclic structure, where the bond between the 2′ carbon and the 3′ carbon in a pentofuranosyl ring is absent.

Modified Internucleoside Linkages

An artificial poly(A) sequence described herein or an RNA polynucleotide containing an artificial poly(A) sequence described herein can contain one or more internucleoside linkages. An internucleoside linkage refers to the backbone linkage that connects the nucleosides. An internucleoside linkage may be a naturally-occurring internucleoside linkage (i.e., a phosphate linkage, also referred to as a 3′ to 5′ phosphodiester linkage, which is found in DNA and RNA) or a modified internucleoside linkage. A modified internucleoside linkage refers to an internucleoside linkage having at least one change that is structurally distinguishable from a naturally-occurring internucleoside linkage. Modified internucleoside linkages may help to improve the stability of an artificial poly(A) sequence described herein or an RNA polynucleotide containing an artificial poly(A) sequence described herein.

Examples of modified internucleoside linkages include, but are not limited to, a phosphorothioate linkage, a phosphorodithioate linkage, a phosphoramidate linkage, a phosphorodiamidate linkage, a thiophosphoramidate linkage, a thiophosphorodiamidate linkage, a phosphoramidate morpholino linkage, and a thiophosphoramidate morpholino linkage, and a thiophosphorodiamidate morpholino linkage, which are known in the art and described in, e.g., Bennett and Swayze, Annu Rev Pharmacol Toxicol. 50:259-293, 2010. A phosphorothioate linkage is a 3′ to 5′ phosphodiester linkage that has a sulfur atom for a non-bridging oxygen in the phosphate backbone of an oligonucleotide. A phosphorodithioate linkage is a 3′ to 5′ phosphodiester linkage that has two sulfur atoms for non-bridging oxygens in the phosphate backbone of an oligonucleotide. A thiophosphoramidate linkage refers to a 3′ to 5′ phospho-linkage that has a sulfur atom for a non-bridging oxygen and a NH group as the 3′-bridging oxygen in the phosphate backbone of an oligonucleotide. In some embodiments, an artificial poly(A) sequence described herein or an RNA polynucleotide containing an artificial poly(A) sequence described herein has at least one (e.g., at least two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, 36, 37, 38, or 39) phosphorothioate linkage. In some embodiments, all of the internucleoside linkages in an artificial poly(A) sequence described herein or an RNA polynucleotide containing an artificial poly(A) sequence described herein are phosphorothioate linkages.

### VI. Methods

The artificial poly(A) sequences described herein can be used in methods of increasing protein expression. The disclosure also provides methods of increasing protein expression of a polypeptide inside a cell by transfecting the cell with an expression vector comprising an expression cassette, wherein the expression cassette comprises a promoter operably linked to a polynucleotide sequence encoding one or more polypeptides and an artificial poly(A) sequence described herein, and wherein the artificial poly(A) sequence is joined to the 3′ end of the polynucleotide sequence. Once the expression vector is transfected into the cell, the one or more polypeptides can be produced from the expression cassette. The RNA polynucleotide comprising the artificial poly(A) sequence is more stable and has longer half-life compared to a corresponding RNA polynucleotide without the artificial poly(A) sequence, which subsequently leads to increased protein expression.

RNA Delivery

In addition to transfecting cells with an expression vector containing an expression cassette such that the RNA polynucleotide comprising the artificial poly(A) sequence can be transcribed inside the cell to produce the protein encoded by the expression vector, an RNA polynucleotide can also be delivered directly into the cells. Examples of RNA delivery systems include, but are not limited to, polymers, exosomes, liposomes, and emulsions. In some embodiments, RNA polynucleotides comprising the artificial poly(A) sequence described herein may be loaded or packaged in liposomes or exosomes that specifically target a cell type, tissue, or organ. For example, exosomes are small membrane-bound vesicles of endocytic origin that are released into the extracellular environment following fusion of multivesicular bodies with the plasma membrane. Exosome production has been described for many immune cells including B cells, T cells, and dendritic cells, Techniques used to load a therapeutic compound (i.e., an RNA polynucleotide comprising the artificial poly(A) sequence) into exosomes are known in the art and described in, e.g., U.S. Patent Publication Nos. US 20130053426 and US 20140348904, and International Patent Publication No. WO 2015002956, which are incorporated herein by reference. In some embodiments, therapeutic compounds may be loaded into exosomes by electroporation or the use of a transfection reagent (i.e., cationic liposomes). In some embodiments, an exosome-producing cell can be engineered to produce the exosome and load it with the therapeutic compound (i.e., an RNA polynucleotide comprising the artificial poly(A) sequence). For example, exosomes may be loaded by transforming or transfecting an exosome-producing host cell with a genetic construct that expresses the therapeutic compound (i.e., an RNA polynucleotide comprising the artificial poly(A) sequence), such that the therapeutic compound is taken up into the exosomes as the exosomes are produced by the host cell.

Various targeting moieties may be introduced into exosomes, so that the exosomes can be targeted to a selected cell type, tissue, or organ. Targeting moieties may bind to cell-surface receptors or other cell-surface proteins or peptides that are specific to the targeted cell type, tissue, or organ. In some embodiments, exosomes have a targeting moiety expressed on their surface. In some embodiments, the targeting moiety expressed on the surface of exosomes is fused to an exosomal transmembrane protein. Techniques of introducing targeting moieties to exosomes are known in the art and described in, e.g., U.S. Patent Publication Nos. US 20130053426 and US 20140348904, and International Patent Publication No. WO 2015002956, which are incorporated herein by reference.

### EXAMPLES

### Example 1—Methods

Table 1 shows the nucleotide sequences of the poly(A) tails used in all the samples. All the poly(A) tails were incorporated into the DNA templates using PCR (Q5® High-Fidelity 2X Master Mix). The purified PCR products were directly used for in vitro synthesis of mRNAs, using standard MEGAscript™ T7 Transcription Kit (Invitrogen). Except the mRNAs described in [0025], all mRNAs were synthesized to contain ARCA cap analog and natural NTPs. All the cells were cultured and passaged using standard media and standard Trypsin protocol. Lipofectamine™ MessengerMax™ (Thermofisher) was used for all the transfections of mRNAs, following the manufacturer's protocol. All the flow cytometry experiments were performed on Attune NxT (Invitrogen). Co-transfection of an iRFP coding mRNA with 120A tail was performed for all experiments and the iRFP intensity from the living cells was used to select positively transfected cells. The analysis of fluorescent intensity from sample mRNAs were performed only on the positively transfected living cell population. The statistical analysis of the data was performed following one-way ANOVA method or using paired-T test.

TABLE 1

| Poly(A) tail | |
|---|---|
| sample name | Sequence (5′ to 3′) |
| 40A | AAAAAAAAAAAAAAAAAAAAAAAAAAAAAAAAAAAAAAAA |
| | (SEQ ID NO: 1) |
| 39AU | AAAAAAAAAAAAAAAAAAAAAAAAAAAAAAAAAAAAAAAU |
| | (SEQ ID NO: 2) |
| 39AC | AAAAAAAAAAAAAAAAAAAAAAAAAAAAAAAAAAAAAAAC |
| | (SEQ ID NO: 3) |
| 39AG | AAAAAAAAAAAAAAAAAAAAAAAAAAAAAAAAAAAAAAAG |
| | (SEQ ID NO: 4) |
| 38ACA | AAAAAAAAAAAAAAAAAAAAAAAAAAAAAAAAAAAAAACA |
| | (SEQ ID NO: 5) |
| 38AGA | AAAAAAAAAAAAAAAAAAAAAAAAAAAAAAAAAAAAAAGA |
| | (SEQ ID NO: 6) |
| 37ACCA | AAAAAAAAAAAAAAAAAAAAAAAAAAAAAAAAAAAAACCA |
| | (SEQ ID NO: 7) |
| 36ACACA | AAAAAAAAAAAAAAAAAAAAAAAAAAAAAAAAAAAACACA |
| | (SEQ ID NO: 8) |
| 35A4CA | AAAAAAAAAAAAAAAAAAAAAAAAAAAAAAAAAAACCCCA |
| | (SEQ ID NO: 9) |
| 31A8CA | AAAAAAAAAAAAAAAAAAAAAAAAAAAAAAACCCCCCCCA |
| | (SEQ ID NO: 10) |
| 23A16CA | AAAAAAAAAAAAAAAAAAAAAAACCCCCCCCCCCCCCCCA |
| | (SEQ ID NO: 11) |
| 30AC9A | AAAAAAAAAAAAAAAAAAAAAAAAAAAAAACAAAAAAAAA |
| | (SEQ ID NO: 12) |
| 20AC19A | AAAAAAAAAAAAAAAAAAAACAAAAAAAAAAAAAAAAAAA |
| | (SEQ ID NO: 13) |
| 10AC29A | AAAAAAAAAACAAAAAAAAAAAAAAAAAAAAAAAAAAAAA |
| | (SEQ ID NO: 14) |
| 60A | AAAAAAAAAAAAAAAAAAAAAAAAAAAAAAAAAAAAAAAA |
| | AAAAAAAAAAAAAAAAAAAA (SEQ ID NO: 15) |
| 55A4CA | AAAAAAAAAAAAAAAAAAAAAAAAAAAAAAAAAAAAAAAA |
| | AAAAAAAAAAAAAAACCCCA (SEQ ID NO: 16) |
| 51A8CA | AAAAAAAAAAAAAAAAAAAAAAAAAAAAAAAAAAAAAAAA |
| | AAAAAAAAAAACCCCCCCCA (SEQ ID NO: 17) |
| 43A16CA | AAAAAAAAAAAAAAAAAAAAAAAAAAAAAAAAAAAAAAAA |
| | AAACCCCCCCCCCCCCCCCA (SEQ ID NO: 18) |
| 100A | AAAAAAAAAAAAAAAAAAAAAAAAAAAAAAAAAAAAAAAA |
| | AAAAAAAAAAAAAAAAAAAAAAAAAAAAAAAAAAAAAAAA |
| | AAAAAAAAAAAAAAAAAAAA (SEQ ID NO: 19) |
| 89A10CA | AAAAAAAAAAAAAAAAAAAAAAAAAAAAAAAAAAAAAAAA |
| | AAAAAAAAAAAAAAAAAAAAAAAAAAAAAAAAAAAAAAAA |
| | AAAAAAAAACCCCCCCCCCA (SEQ ID NO: 20) |
| 10C_{scramble} | AAAAAAAAAAAAAAAAAAAAAAAAAAAAAAAAAAAAAAAA |
| | AAAAAAAAAAAAAAAAAAAAAAAAAAAAAACAAACCAAAA |
| | CACACAAAAAACACCAAACA (SEQ ID NO: 21) |
| 79A20CA | AAAAAAAAAAAAAAAAAAAAAAAAAAAAAAAAAAAAAAAA |
| | AAAAAAAAAAAAAAAAAAAAAAAAAAAAAAAAAAAAAAAC |
| | CCCCCCCCCCCCCCCCCCCA (SEQ ID NO: 22) |
| 69A30CA | AAAAAAAAAAAAAAAAAAAAAAAAAAAAAAAAAAAAAAAA |
| | AAAAAAAAAAAAAAAAAAAAAAAAAAAAACCCCCCCCCCCC |
| | CCCCCCCCCCCCCCCCCCA (SEQ ID NO: 23) |
| 120A | AAAAAAAAAAAAAAAAAAAAAAAAAAAAAAAAAAAAAAAA |
| | AAAAAAAAAAAAAAAAAAAAAAAAAAAAAAAAAAAAAAAA |
| | AAAAAAAAAAAAAAAAAAAAAAAAAAAAAAAAAAAAAAAA |
| | (SEQ ID NO: 24) |
| 38ACC | AAAAAAAAAAAAAAAAAAAAAAAAAAAAAAAAAAAAAACC |
| | (SEQ ID NO: 25) |

### Example 2—Cytidine Substitution Near the End of Poly(A) Tail can Significantly Enhance Protein Expression of mRNA

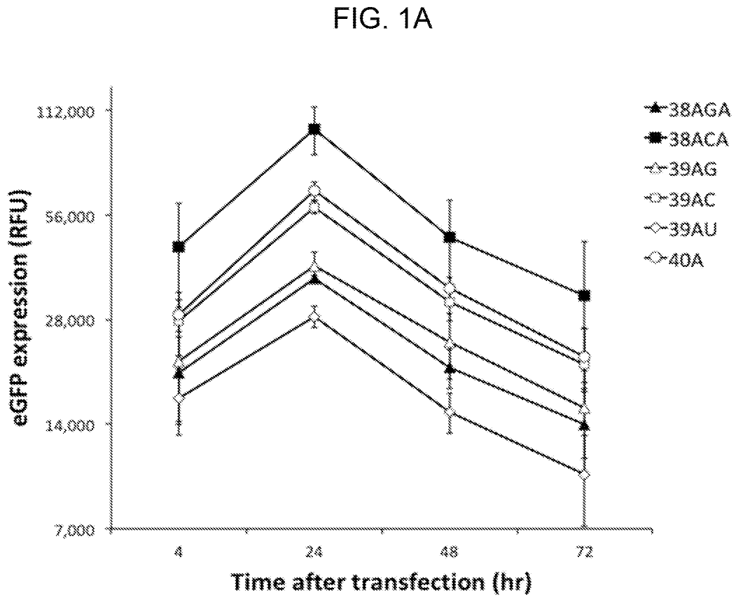

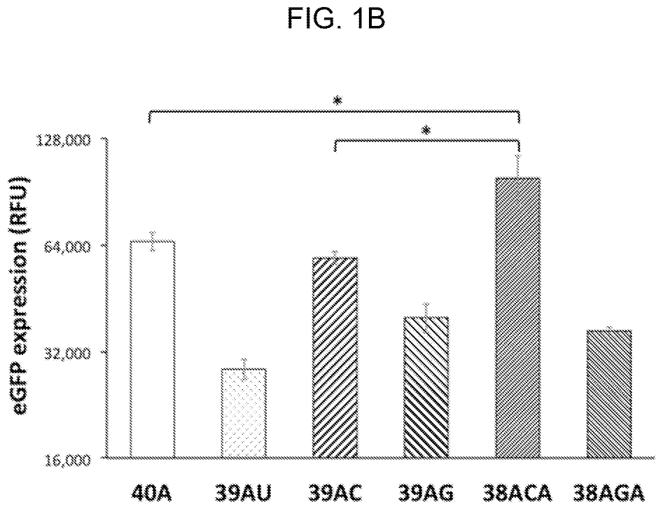

A series of EGFP coding mRNAs was synthesized. Each mRNA carried a different single nucleotide substitution at the last or second last position of the poly(A) tail. An EGFP mRNA carrying a native fourth adenine tail (40A poly(A) tail) served as reference. The EGFP expression from HEK293 cells was observed using flow cytometer after 4, 24, 48, and 72 hours after mRNA transfection. It was found that EGPF mRNA with 38ACA poly(A) tail, having a single cytidine substitution at the second last nucleotide, exhibited the highest expression of EGFP at all the time points (FIG. 1A). Statistical analysis of the EGFP expression at 24 hours, which was when the peak expression occurred, revealed that the mRNA with 38ACA poly(A) tail provided significantly higher EGFP expression (FIG. 1i). This data shows cytidine substitution near the end of poly(A) tail has a positive influence on the protein expression of mRNAs.

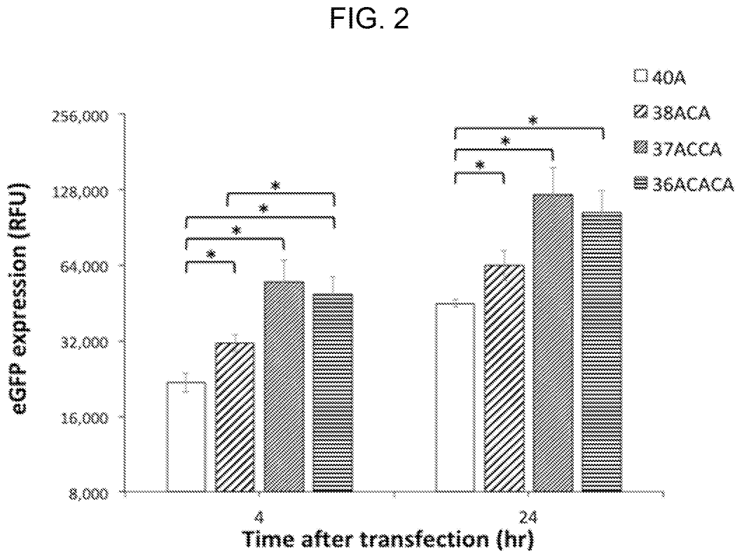

Dual cytidine substitution near the end of poly(A) tail was tested next, with two adjacent cytidines: sample 37ACCA, and two separate cytidines: sample 36ACACA. Repeating the previous experiment on HEK293, it was discovered that the EGFP mRNAs with both types of dual cytidine substitutions exhibited higher protein expression level, observed after 4 and 24 hours of transfection (FIG. 2).

### Example 3—Effect of Cytidine Substitution is Cell Type Independent

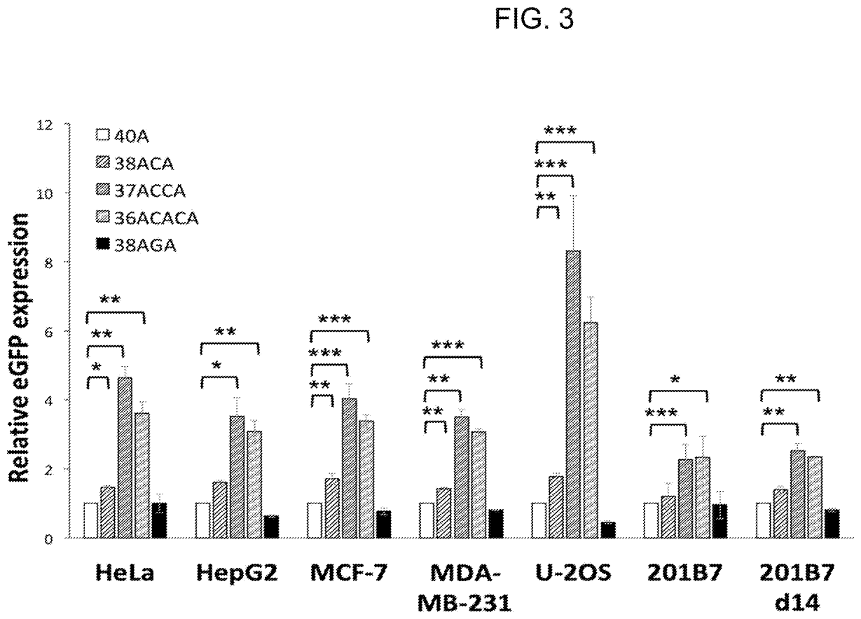

The EGFP expressions from EGFP mRNAs that carry different poly(A) tails on several types of culture human cells were compared after 24 hours of transfection. HeLa cell was used because it is a common model cell. Several cancer cell lines were selected because they have different tissue origins: HepG2 (liver), MCF-7 (breast), MDA-MB-231 (breast), and U-20S (bone). An induced human pluripotent stem cell (iPSC) 201B7 was chosen because it is a well-established iPSC strain that originated from healthy adults. Randomly differentiated version of 201B7 cells: 201B7d14 (obtained after 14 days of culture in culture media without bFGF) was also chosen to represent a mixture of healthy somatic cells [20]. As shown in FIG. 3, on all the cell lines tested, EGFP mRNAs with cytidine(s) near the end of their poly(A) tails exhibited significantly higher EGFP expression.

### Example 4—Effective Cytidine Substitution Frequency

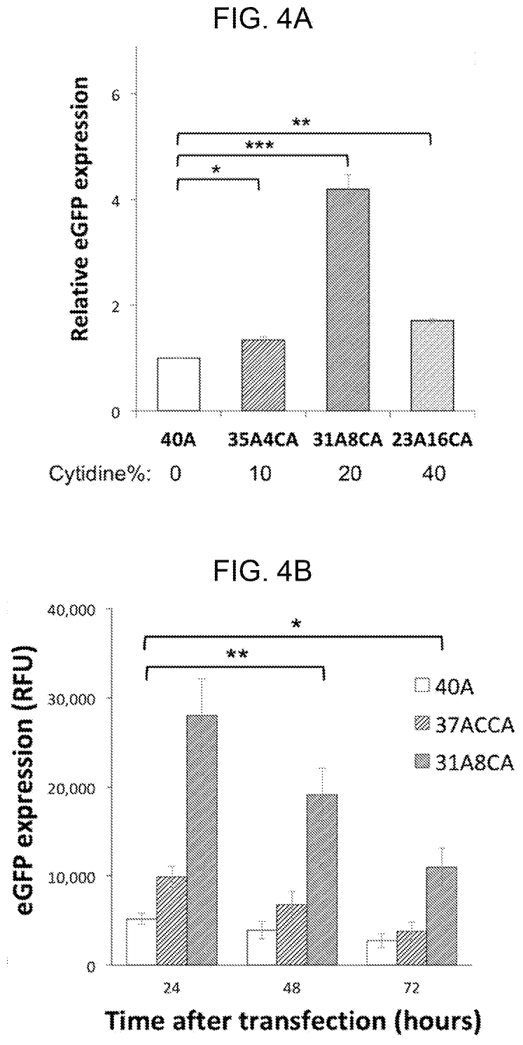

The EGFP expressions from EGFP mRNAs that carry different numbers of cytidine substitutions (adjacent substitution from the second last nucleotide towards the 5′ end) on the poly(A) tails on HEK293 cells were compared after 24 hours of transfection. FIG. 4A shows that while the mRNAs having poly(A) tail samples with 10% and 40% of cytidine exhibited significantly higher eGPF expression, the EGFP mRNA with 31A8CA poly(A) tail, that is 20% of cytidine, exhibited the strongest EGFP expression. By tracking the expression over time, significantly higher EGFP expression for three days from HEK293 cells transfected with EGFP mRNA with 31A8CA poly(A) tail (FIG. 4B) was observed. The expression from mRNA with 31A8CA poly(A) tail at 72 hours was even higher than that from mRNA with 40A poly(A) tail at 24 hours. It seems that the peak of expression enhancement from cytidine substitution lies around 20% of cytidine substitution, and the whole effective range is from single substitution to 40% substitution.

### Example 5—Effective Cytidine Substitution Location is in Rear Part of the Poly(A) Tail, not Including the Last Nucleotide Position

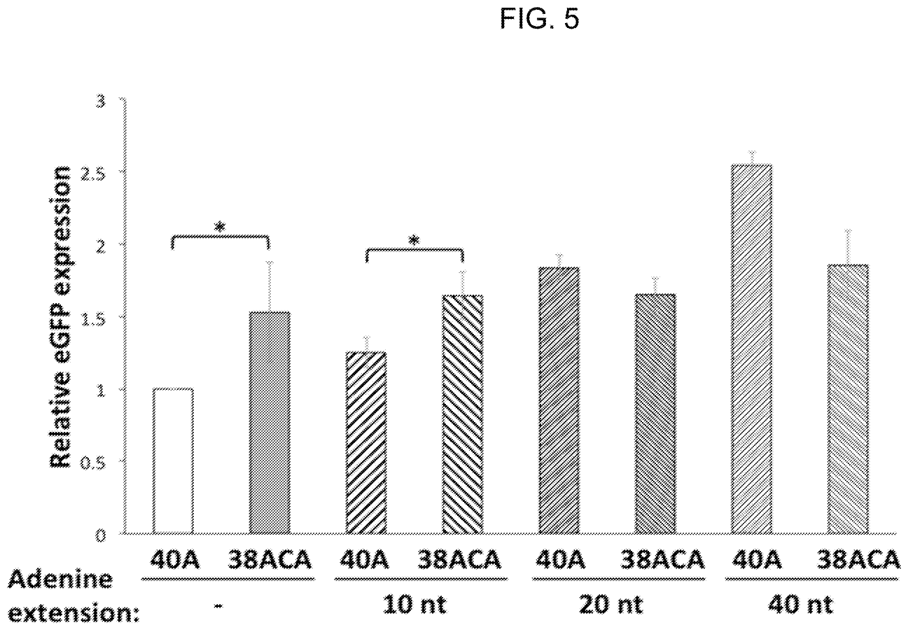

E. coli Poly(A) Polymerase (NEB) was used to extend extra adenines at the end of EGFP mRNAs carrying 40A and 38ACA poly(A) tail [21]. By controlling the reaction time, we estimated the number of adenines added based on the unit definition of the enzyme provided by the supplier. By transfecting these mRNAs into HEK293 cells, the enhancement of EGFP expressions from EGFP mRNA with 38ACA poly(A) tail and EGFP mRNA with 38ACA poly(A) tail plus 10 nt adenines (FIG. 5) was observed. This suggests that protein expression enhancement only occurs when the cytidine substitution resides in the rear part of the poly(A) tail (about last 40%). The single nucleotide substitution data in FIGS. 1A and 1B suggest that cytidine substitution on the last nucleotide has no positive effect on protein expression of mRNA.

### Example 6—Cytidine Substitution can Enhance the Performance of Simple and Smart mRNA Drug

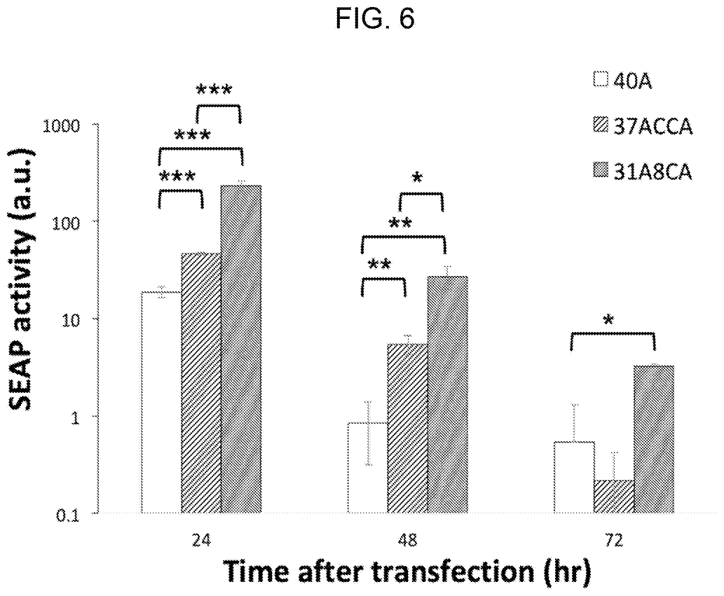

An mRNA that encoded a functional protein SEAP was constructed as a model of simple protein delivery mRNA drug [22]. Culture medium was completely collected after 24 hours of transfecting SEAP mRNAs to HEK293cells. The cells were immediately supplied with equal volume of fresh medium and the medium was completely collected again at 48 hours after transfection. The cells were immediately supplied with equal volume of fresh medium and the medium was collected again at 72 hours after transfection. The activity of SEAP in each collected culture medium was quantified using the Alkaline Phosphatase Activity Fluoremetric Assay kit (BioVision) [23]. It was found that there was a strong positive correlation between the activity level of SEAP and the frequency of cytidine substitution near the end of poly(A) tail (FIG. 6). Cells transfected with SEAP mRNA with 31A8CA poly(A) tail was able to maintain a significantly higher SEAP activity over three days comparing with that transfected with SEAP mRNA with 40A poly(A) tail.

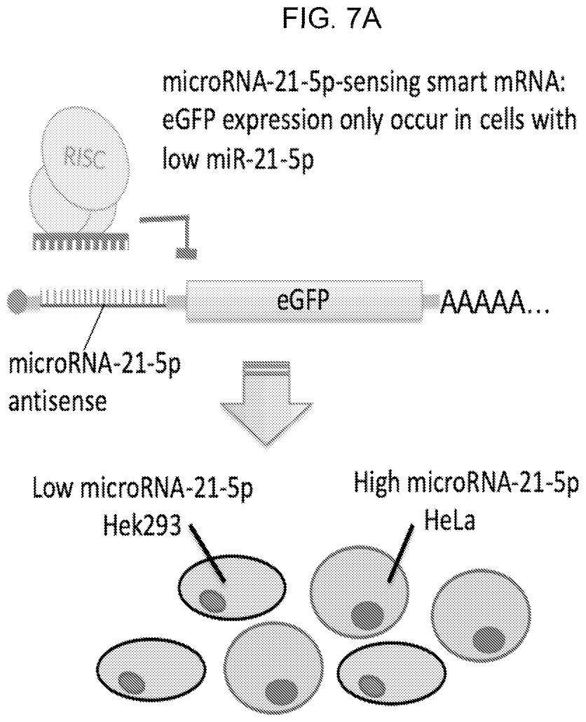

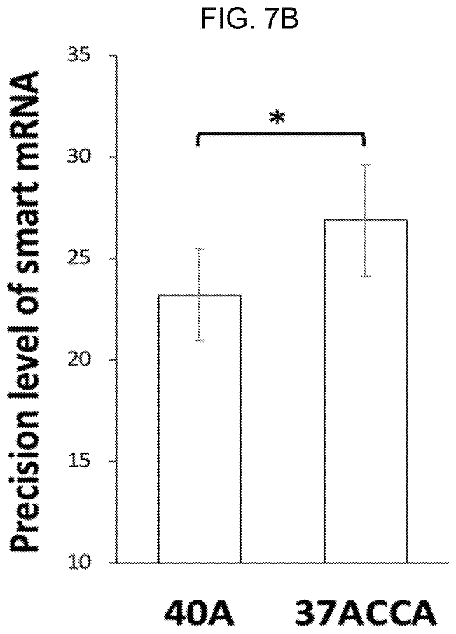

mRNAs that carry two copies of microRNA-21-5p antisense sequence motif in the 5′UTR and encode EGFP were constructed as a model of smart mRNA drugs that have targeted protein delivery ability. The EGFP expression from these mRNAs was inhibited by microRNA-21-5p mediated mRNA suppression. A co-culture condition was created by seeding a mixture of high microRNA-21-5p expressing HeLa cells and low microRNA-21-5p expressing HEK293 cells. After 24 hours of transfection, the separation of the two cell populations based on EGFP expression from the sample mRNAs and the iRFP expression of reference iRFP mRNA was recorded. It was found that the degree of separation, denoted as precision level, was more obvious when the mRNA carries 37ACCA poly(A) tail (FIGS. 7A and 7B).

### Example 7—Cytidine Substitution does not Affect Cell Viability, Transfection Efficiency, and Prolongs Intracellular mRNA Half-Life

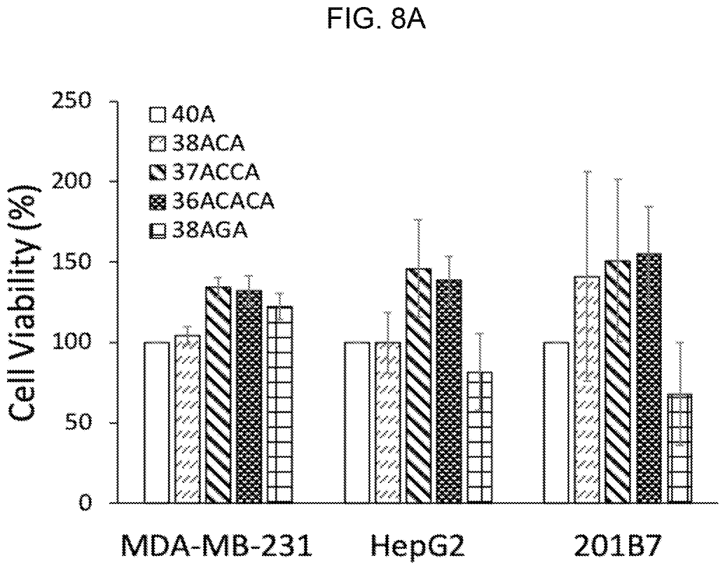

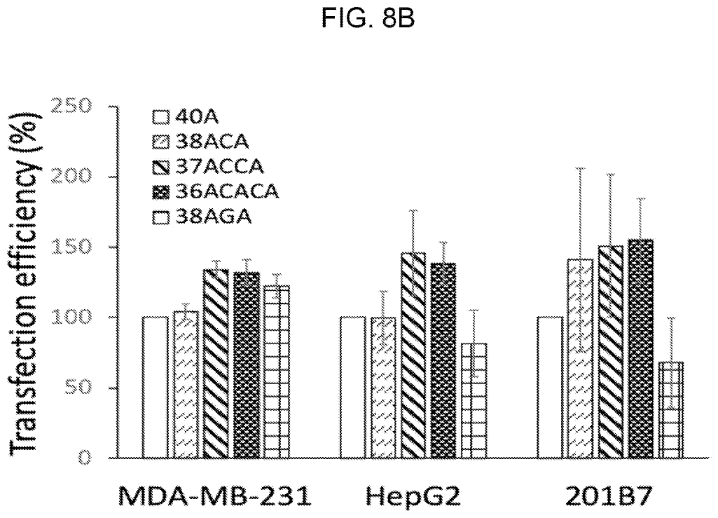

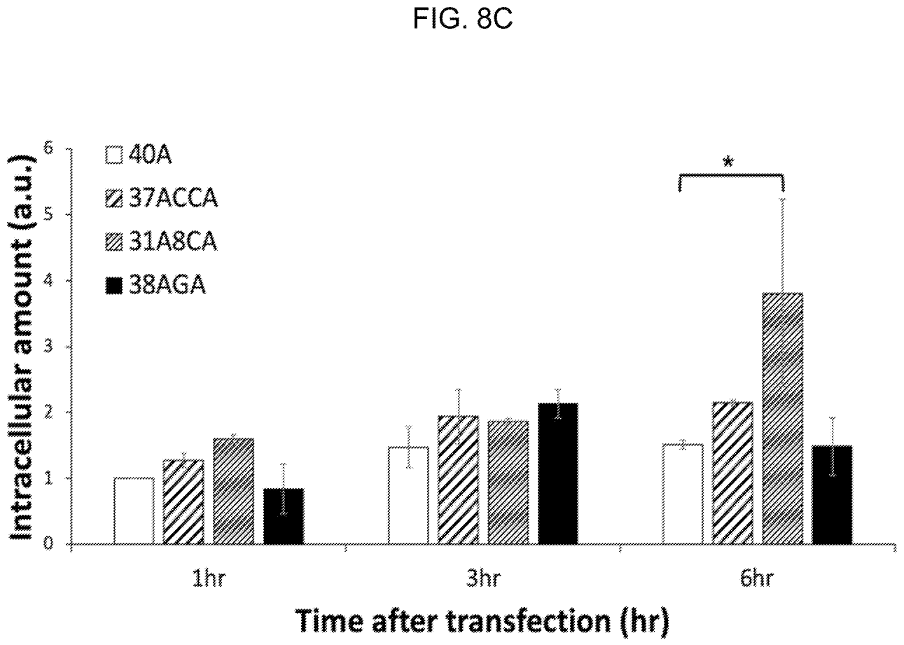

Cell viability of several types of cells was quantified by counting the total number of viable cells after 24 hours of transfection of EGFP mRNAs with different poly(A) tails. Further, mRNA transfection efficiency was quantified by comparing the number of EGFP positive viable cells against the total number of viable cells after 24 hours of transfection of EGFP mRNAs with different poly(A) tails. No significant difference in either cell viability or transfection efficiency among cells transfected with these mRNAs was observed. The amount of EGFP mRNAs inside HEK293 cells after 3, 6, and 12 hours after transfection was quantified using RT-qPCR with the baseline subtraction method. The amount of 18S rRNA was used for normalization [24]. FIGS. 8A-8C show that after 6 and 12 hours of transfection, there was clearly more EGFP mRNA with 37ACCA poly(A) tail remaining inside the cells, suggesting these mRNAs have longer intracellular half-lives.

### Example 8—Effect of Cytidine Substitution on Poly(A) Tail of EGFP mRNA with Different Lengths

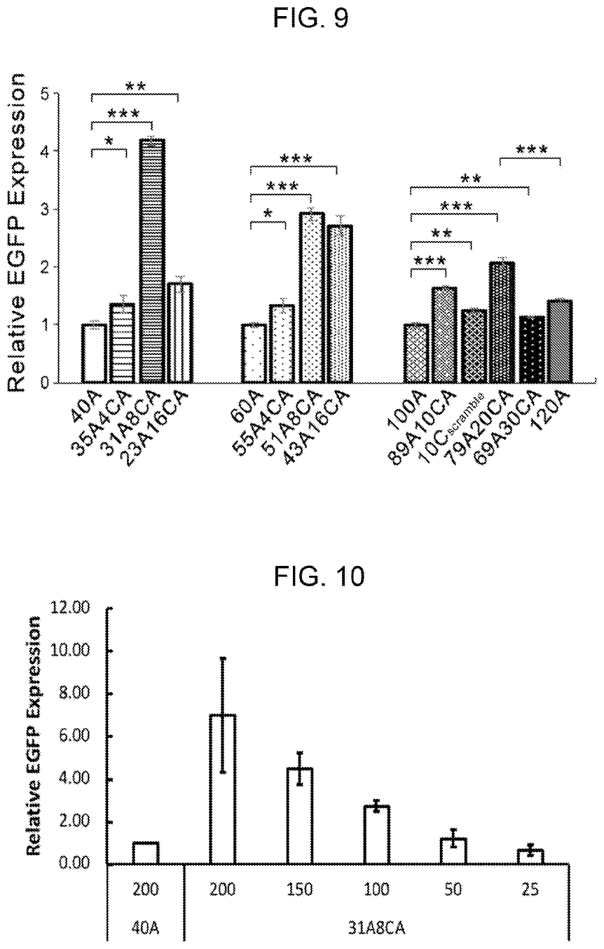

A 100 nt tail with scrambled C insertion in the last 30% of the tail was also tested. Such random and discontinued C insertion also has weak effect on protein expression enhancement (FIG. 9). A commonly used tail with 120A was also included as a reference. As can be seen in FIG. 9, the effect of cytosine substitution on increasing protein expression works with poly(A) tail of different lengths.

### Example 9—Lower Concentration of mRNA with C Tail can Achieve Similar or Higher Protein Expression Level as mRNA with A-Only Tail

HEK293 cells in 48-well plate were transfected with EGFP-40A mRNA at 200 ng/mL or EGFP-31A8CA mRNA at 25 ng/mL to 200 ng/mL. As can be seen in FIG. 10, even transfecting EGFP-31A8CA mRNA at the lowest amount, 25 ng/mL, the expression of EGFP was similar to transfecting EGFP-40A mRNA at 200 ng/mL, 8 times the amount of EGFP-31A8CA mRNA.

### Example 10—the Effect of C Tails is Independent to Transfection Reagent

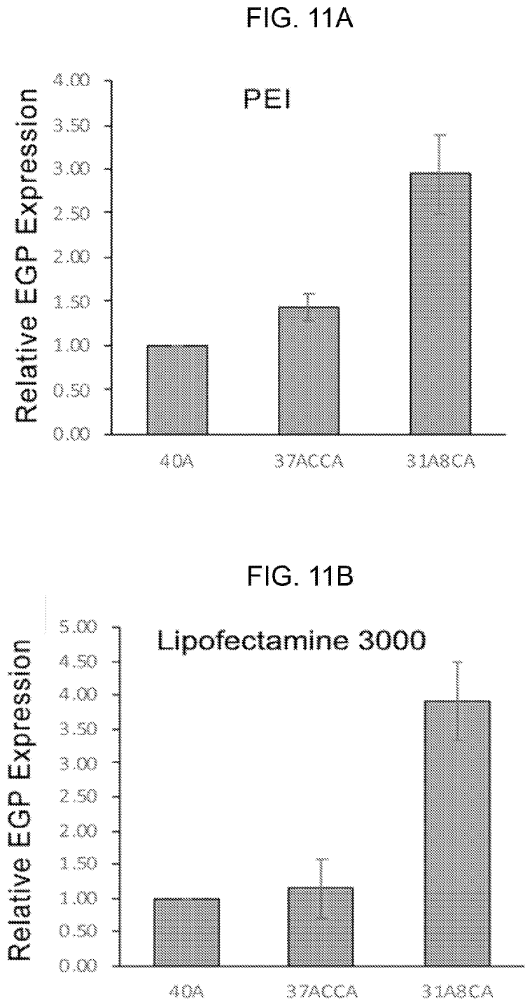

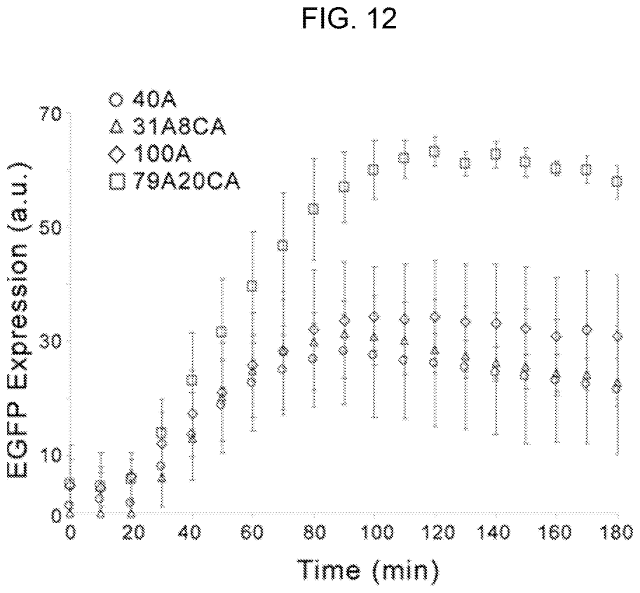

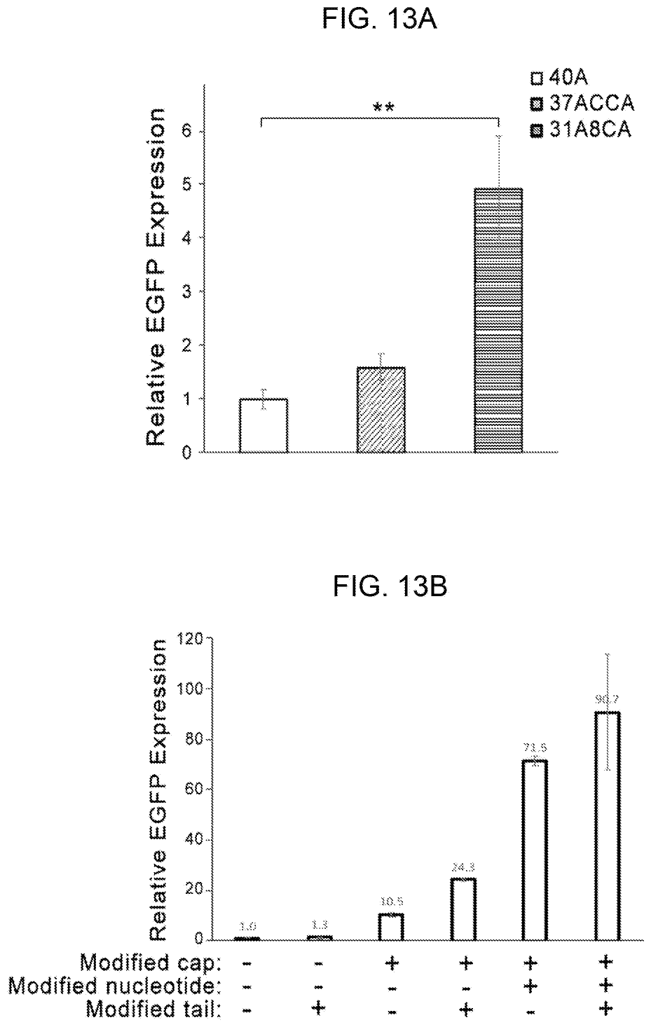

HEK293 cells in 48-well plate were transfected with EGFP-40A, EGFP-37ACCA, or EGFP-31A8CA mRNAs using PEI or lipofectamine 3000 reagents. As can be seen in FIGS. 11A and 111B, after 24 hours of transfection, the enhancement of EGFP with C tails were observed in both cases.

### Example 11—the C Tails can Enhance Protein Production from mRNAs in Cell Lysate

1 μg of EGFP-40A, EGFP-31A8CA, EGFP-100A, or EGFP-79A20CA mRNAs and 1 μL RNase inhibitor were mixed into nuclease-free water to make 10 μL diluted mRNA solutions. The diluted mRNA solutions were heated to 70° C. for 3 minutes on heat block then immediately incubated on ice for more than 1 minutes. 50 μL reaction mixture containing 1× translation mix minus-Methionine, 0.5 μM Methionine, 10 μL diluted mRNA solution and 35 μL of HeLa Cytoplasmic Extracts were prepared in black 96-well plate. The reaction mixtures were incubated at 30° C. for 3 hours in FlexStation 3. EGFP productions were monitored through fluorescence measurement (Ex/Em: 480/520; cutoff: 495 nm) at an interval of 10 minutes. As can be seen in FIG. 12, EGFP-79A20CA mRNA had clearly higher EGFP production than EGFP-100A and EGFP-31A8CA mRNA had slightly higher EGFP production than EGFP-40A at transition phase (60 to 120 mins).

### Example 12—the C Tails Used Together with Other mRNA Technologies can Achieve Synergic Effect for Boosting of Protein Production from mRNA

HEK293 cells in 48-well plate were transfected with EGFP mRNAs carrying different types of mRNA enhancement technologies. After 24 hours of transfection, the EGFP production from the cells was recorded for comparison. C tail carrying EGFP mRNAs with modified cap analog exhibited higher protein production than the EGFP mRNA with only C tail or only modified cap analog. C tail carrying EGFP mRNAs with modified cap analog and modified nucleotides exhibited higher protein production than the EGFP mRNA with only C tail and modified cap analog.

### Example 13—the C Tails can Contain C as the Last Nucleotide when the Tail Contains Multiple C Substitution

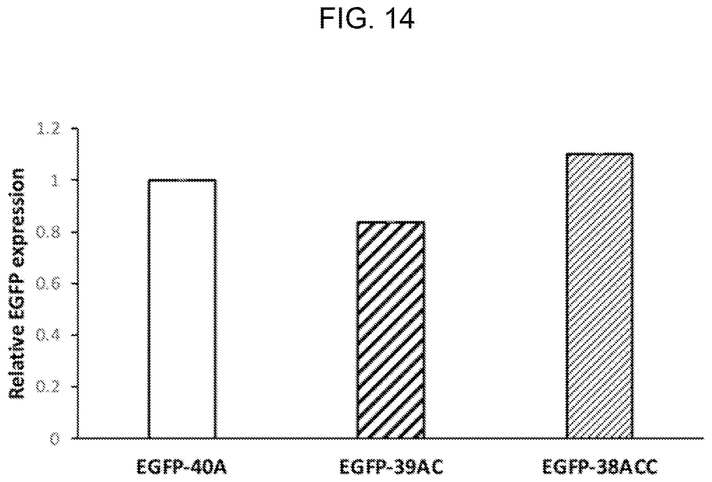

HEK293 cells in 48-well plate were transfected with EGFP-40A, EGFP-39AC or EGFP-38A8CC mRNAs. As can be seen in FIG. 14, after 24 hours of transfection, EGFP-38A8CC produced higher EGFP protein then EGFP-40A.

### REFERENCES

[1] U. Sahin, K. Karikó, and Ö. Türeci, “mRNA-based therapeutics-developing a new class of drugs,” Nature Reviews Drug discovery, vol. 13, no. 10, p. 759, 2014.
[2] N. Pardi, M. J. Hogan, F. W. Porter, and D. Weissman, “mRNA vaccines-a new era in vaccinology,” Nature reviews Drug discovery, vol. 17, no. 4, p. 261, 2018.
[3] T. Schlake, A. Thess, M. Fotin-Mleczek, and K.-J. Kallen, “Developing mRNA-vaccine technologies,” RNA biology, vol. 9, no. 11, pp. 1319-1330, 2012.
[4] J. A. Wolff et al., “Direct gene transfer into mouse muscle in vivo,” Science, vol. 247, no. 4949, pp. 1465-1468, 1990.
[5] G. F. Jirikowski, P. P. Sanna, D. Maciejewski-Lenoir, and F. E. Bloom, “Reversal of diabetes insipidus in Brattleboro rats: intrahypothalamic injection of vasopressin mRNA,” Science, vol. 255, no. 5047, pp. 996-998, 1992.
[6] F. Martinon et al., “Induction of virus-specific cytotoxic T lymphocytes in vivo by liposome-entrapped mRNA,” European Journal of Immunology, vol. 23, no. 7, pp. 1719-1722, 1993.
[7] C. W. Mandl, J. H. Aberle, S. W. Aberle, H. Holzmann, S. L. Allison, and F. X. Heinz, “In vitro-synthesized infectious RNA as an attenuated live vaccine in a flavivirus model,” Nature Medicine, vol. 4, no. 12, pp. 1438-1440, 1998.
[8] W.-Z. Zhou et al., “RNA melanoma vaccine: induction of antitumor immunity by human glycoprotein 100 mRNA immunization,” Human Gene Therapy, vol. 10, no. 16, pp. 2719-2724, 1999.
[9] M. S. Kormann et al., “Expression of therapeutic proteins after delivery of chemically modified mRNA in mice,” Nature Biotechnology, vol. 29, no. 2, pp. 154-157, 2011.
[10] M. Mockey, C. Gonçalves, F. P. Dupuy, F. M. Lemoine, C. Pichon, and P. Midoux, “mRNA transfection of dendritic cells: synergistic effect of ARCA mRNA capping with Poly (A) chains in cis and in trans for a high protein expression level,” Biochemical Biophysical Research Communications, vol. 340, no. 4, pp. 1062-1068, 2006.
[11] K. Karikó, “In vitro-Transcribed mRNA Therapeutics: Out of the Shadows and Into the Spotlight,” Molecular Therapy, vol. 27, no. 4, pp. 691-692, 2019.
[12] K. Karikó et al., “Incorporation of pseudouridine into mRNA yields superior nonimmunogenic vector with increased translational capacity and biological stability,” Molecular therapy, vol. 16, no. 11, pp. 1833-1840, 2008.
[13] K. Karikó, M. Buckstein, H. Ni, and D. Weissman, “Suppression of RNA recognition by Toll-like receptors: the impact of nucleoside modification and the evolutionary origin of RNA,” Immunity, vol. 23, no. 2, pp. 165-175, 2005.
[14] C. J. C. Parr et al., “N1-Methylpseudouridine substitution enhances the performance of synthetic mRNA switches in cells,” Nucleic Acids Research, vol. 48, no. 6, pp. e35-e35, 2020.
[15] E. Hajnsdorf and V. R. Kaberdin, “RNA polyadenylation and its consequences in prokaryotes,” Philosophical Transactions of the Royal Society B: Biological Sciences, vol. 373, no. 1762, p. 20180166, 2018.
[16] H. Chang, J. Lim, M. Ha, and V. N. Kim, “TAIL-seq: genome-wide determination of poly (A) tail length and 3′ end modifications,” Molecular Cell, vol. 53, no. 6, pp. 1044-1052, 2014.
[17] D. Zheng and B. Tian, “Sizing up the poly (A) tail: insights from deep sequencing,” Trends in Biochemical Sciences, vol. 39, no. 6, pp. 255-257, 2014.
[18] D. Strzelecka, M. Smietanski, P. Sikorski, M. Warminski, J. Kowalska, and J. Jemielity, “Functional and LC-MS/MS analysis of in vitro transcribed mRNAs carrying phosphorothioate or boranophosphate moieties reveal polyA tail modifications that prevent deadenylation without compromising protein expression,” bioRxiv, 2020.
[19] J.-D. Beaudoin and J.-P. Perreault, “Exploring mRNA 3′-UTR G-quadruplexes: evidence of roles in both alternative polyadenylation and mRNA shortening,” Nucleic Acids Research, vol. 41, no. 11, pp. 5898-5911, 2013.
[20] M. Nakagawa et al., “A novel efficient feeder-free culture system for the derivation of human induced pluripotent stem cells,” Scientific Reports, vol. 4, p. 3594, 2014.
[21] S. Yehudai-Resheff and G. Schuster, “Characterization of the E. coli poly(A) polymerase: nucleotide specificity, RNA-binding affinities and RNA structure dependence,” Nucleic Acids Research, vol. 28, no. 5, pp. 1139-1144, 2000.
[22] J. Berger, J. Hauber, R. Hauber, R. Geiger, and B. R. Cullen, “Secreted placental alkaline phosphatase: a powerful new quantitative indicator of gene expression in eukaryotic cells,” Gene, vol. 66, no. 1, pp. 1-10, 1988.
[23] H. D. Holscher, S. R. Davis, and K. A. Tappenden, “Human milk oligosaccharides influence maturation of human intestinal Caco-2Bbe and HT-29 cell lines,” The Journal of Nutrition, vol. 144, no. 5, pp. 586-591, 2014.
[24] K. Goossens, M. Van Poucke, A. Van Soom, J. Vandesompele, A. Van Zeveren, and L. J. Peelman, “Selection of reference genes for quantitative real-time PCR in bovine preimplantation embryos,” BMC Developmental Biology, vol. 5, no. 1, pp. 1-9, 2005.

The above examples are provided to illustrate the disclosure but not to limit its scope. Other variants of the disclosure will be readily apparent to one of ordinary skill in the art and are encompassed by the appended claims. All publications, databases, internet sources, patents, patent applications, and accession numbers cited herein are hereby incorporated by reference in their entireties for all purposes.

1. An artificial poly(A) sequence comprising about 30-150 adenines, wherein at least one adenine is substituted with a cytosine in the last one-third portion of the artificial poly(A) sequence closest to its 3′ end.

2. The artificial poly(A) sequence of claim 1, wherein the artificial poly(A) sequence comprises between 18 and 129 adenines.

3. The artificial poly(A) sequence of claim 1, wherein the last nucleotide of the artificial poly(A) sequence is not a cytosine.

4. The artificial poly(A) sequence of claim 1, wherein up to 40% of the nucleotides in the artificial poly(A) sequence are cytosines.

5. The artificial poly(A) sequence of claim 4, wherein up to 25% of the nucleotides in the artificial poly(A) sequence are cytosines.

6. The artificial poly(A) sequence of claim 1, wherein most of the cytosines in the artificial poly(A) sequence are located in the last one-third portion of the artificial poly(A) sequence closest to its 3′ end.

7. The artificial poly(A) sequence of claim 1, wherein all of the cytosines in the artificial poly(A) sequence are located consecutively.

8. The artificial poly(A) sequence of claim 1, wherein the artificial poly(A) sequence comprises about 40 adenines and at least one adenine is substituted with a cytosine between the 27^{th} nucleotide and the 39^{th} nucleotide of the artificial poly(A) sequence.

9. The artificial poly(A) sequence of claim 8, wherein the artificial poly(A) sequence comprises between 24 and 39 adenines.

10. The artificial poly(A) sequence of claim 8, wherein the artificial poly(A) sequence comprises between 1 and 16 cytosines.

11. The artificial poly(A) sequence of claim 8, wherein all of the cytosines in the artificial poly(A) sequence are located between the 25^{th} nucleotide and the 39^{th} nucleotide of the artificial poly(A) sequence.

12. The artificial poly(A) sequence of claim 8, wherein all of the cytosines in the artificial poly(A) sequence are located consecutively.

13. The artificial poly(A) sequence of claim 8, wherein the last nucleotide of the artificial poly(A) sequence is not a cytosine.

14. The artificial poly(A) sequence of claim 1, wherein the artificial poly(A) sequence comprises about 60 adenines and at least one adenine is substituted with a cytosine between the 41^{st} nucleotide and the 59^{th} nucleotide of the artificial poly(A) sequence.

15. The artificial poly(A) sequence of claim 14, wherein the artificial poly(A) sequence comprises between 36 and 59 adenines.

16. The artificial poly(A) sequence of claim 14, wherein the artificial poly(A) sequence comprises between 1 and 24 cytosines.

17. The artificial poly(A) sequence of claim 14, wherein all of the cytosines in the artificial poly(A) sequence are located between the 37^{th} nucleotide and the 59^{th} nucleotide of the artificial poly(A) sequence.

18. The artificial poly(A) sequence of claim 14, wherein all of the cytosines in the artificial poly(A) sequence are located consecutively.

19. The artificial poly(A) sequence of claim 14, wherein the last nucleotide of the artificial poly(A) sequence is not a cytosine.

20. The artificial poly(A) sequence of nm claim 1, wherein the artificial poly(A) sequence comprises about 100 adenines and at least one adenine is substituted with a cytosine between the 67^{th} nucleotide and the 99^{th} nucleotide of the artificial poly(A) sequence.

21. The artificial poly(A) sequence of claim 20, wherein the artificial poly(A) sequence comprises between 60 and 99 adenines.

22. The artificial poly(A) sequence of claim 20, wherein the artificial poly(A) sequence comprises between 1 and 40 cytosines.

23. The artificial poly(A) sequence of claim 20, wherein all of the cytosines in the artificial poly(A) sequence are located between the 61^{st} nucleotide and the 99^{th} nucleotide of the artificial poly(A) sequence.

24. The artificial poly(A) sequence of claim 20, wherein all of the cytosines in the artificial poly(A) sequence are located consecutively.

25. The artificial poly(A) sequence of claim 20, wherein the last nucleotide of the artificial poly(A) sequence is not a cytosine.

26. An expression cassette comprising a promoter and a polynucleotide sequence encoding the artificial poly(A) sequence of claim 1.

27. The expression cassette of claim 26, further comprising a multiple cloning site between the promoter and the polynucleotide sequence encoding the artificial poly(A) sequence.

28. The expression cassette of claim 26, further comprising a transcription initiation codon and a transcription termination codon, both operably linked to the promoter and the polynucleotide sequence encoding the artificial poly(A) sequence.

29. The expression cassette of claim 26, further comprising a polynucleotide sequence encoding a polypeptide between the promoter and the artificial poly(A) sequence, wherein the polynucleotide sequence is operably linked to the promoter and the polynucleotide sequence encoding the artificial poly(A) sequence.

30. An expression vector comprising the expression cassette of claim 26.

31. A host cell comprising the expression cassette of claim 26.

32. An RNA polynucleotide expressed from the expression cassette of claim 29.

33. An RNA molecule comprising a coding sequence for a polypeptide and the artificial poly(A) sequence of claim 1.

34. A method of increasing protein expression of a polypeptide inside a cell, comprising transfecting the cell with the expression vector of claim 30.
